(19) United States
(12) Patent Application Publication
(10) Pub. No.: US 2022/0249747 A1
(43) Pub. Date: Aug. 11, 2022

(54) EAR CANAL EVALUATION METHOD

(71) Applicant: King Abdulaziz University, Jeddah (SA)

(72) Inventor: Mohammed AL HADDAD, Jeddah (SA)

(73) Assignee: King Abdulaziz University, Jeddah (SA)

(21) Appl. No.: 17/206,332

(22) Filed: Mar. 19, 2021

Related U.S. Application Data
(63) Continuation of application No. 17/171,659, filed on Feb. 9, 2021.

(51) Int. Cl. A61M 1/00

(52) U.S. Cl. CPC A61M 1/0039; A61M 1/0031; A61M 2205/3331; A61M 2205/273; A61M 2210/0662; A61M 2205/587

(57) ABSTRACT

An ear suctioning device having a suction tube and a disposable hollow tip is provided. The suction tube has a distal segment and a proximal segment which are angled with respect to each other at about 130 to 170 degrees. The disposable hollow tip is reversibly connected to a distal end of the suction tube and fluidly connected to the suction tube. The disposable hollow tip comprises a safety flange which prevents the suction tube from contacting an ear or ear canal into which the ear suctioning device is inserted. A pressure-equalizing hole is disposed on the proximal segment of the suction tube and in fluid communication with an interior volume of the suction tube. A proximal end of the suction tube is attached to a vacuum or low pressure line.

### BACKGROUND OF THE INVENTION

### Field of the Invention

The present disclosure relates to ear or ear canal cleaning and, more particularly relates to an ear suctioning device and a method of providing suction to an ear canal for performing cleaning operation in the ear canal.

### Discussion of the Background

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

Ear, nose and throat (otherwise referred as ENT) clinics are some of the busiest clinics around the world. Common diseases seen in those clinics include infections of middle ear and external ear, and the presence of wax, dirt and foreign bodies in the external ear canal. One of the most common medical interventions performed by otorhinolaryngologists and some clinicians is micro-suction and manual clearance of the ears by using suction tubes. The frequency of use of this medical intervention may substantially exceed half the number of patients in a general ENT clinic per day. Suction tubes are very important medical instruments used to clean the wax, discharges and debris from the ears.

The conventional suction tube is generally a metal piece with two ends; one end is connected to a suction device and another end is used inside the ear to clean the ear canal. However, the existing suction tubes are associated with some complications and shortcomings. One problem associated with the existing suction tubes is the transmission of bacterial and fungal infections from one ear to another ear of the same patient or from the ears of one patient to another patient. Another common issue is lacerations and cut injuries to the external ear, ear canal wall, and the middle ear caused by the suction tube itself. Such injuries range in severity from minor temporary pain to permanent damage including eardrum perforation, especially in pediatric patients and adult patients who have uncontrolled reactions while performing cleaning operation in the ear or ear canal. In some circumstances, due to over reactions and non-cooperation from patients, the patients are referred to a day surgery under general anesthesia to avoid complications that may be otherwise caused by the existing suction tubes.

Further, the existing suction tubes are associated with other problems such as hazards of frequent sterilization and manual cleaning, frequent blockage of the suction tubes due to narrow lumen, risks and difficulties of using metallic, hard and sharp ordinary suction tubes by junior physicians during training programs, and difficulties associated with gripping small narrow suction tubes by hand.

Hence, there remains a need to develop a suction tube that can overcome aforementioned shortcomings and problems and perform ear cleaning with greater safety and ease of use even by a junior physician.

### SUMMARY OF THE INVENTION

The present disclosure relates to an ear suctioning device. The ear suctioning device comprises a suction tube having a distal segment and a proximal segment which are angled with respect to each other at about 130 to 170 degrees. A hollow tip is reversibly connected to a distal end of the suction tube and fluidly connected to the suction tube. A pressure-equalizing hole is disposed on the proximal segment and in fluid communication with an interior volume of the suction tube. A proximal end of the suction tube is configured to attach to a vacuum or low pressure line. The hollow tip comprises a safety flange which prevents the suction tube from contacting an ear or ear canal into which the ear suctioning device is inserted.

In some embodiments, the pressure-equalizing hole is located in a finger cup having a substantially hemiovoid shape and is configured such that the pressure-equalizing hole is capable of being partially or totally occluded by a finger or thumb placed in the finger cup.

In some embodiments, the ear suctioning device comprises an ergonomic grip located on or attached to the proximal segment.

In some embodiments, the ear suctioning device comprises a controllable pressure-equalizing hole occlusion mechanism.

In some embodiments, the suction tube comprises a biocompatible metal.

In some embodiments, the ear suctioning device comprises a tip interlock comprising two or more interlock components, and at least one of which is located on each of the suction tube and the hollow tip.

In some embodiments, the hollow tip comprises a flexible free end and a non-flexible fixed end.

In some embodiments, the flexible free end comprises a silicone.

In some embodiments, the hollow tip is configured to be disposable.

In some embodiments, the non-flexible fixed end comprises a projection portion which is configured to fit securely into the distal segment of the suction tube.

In some embodiments, the projection portion has a projection portion length which is 25 to 75% of a total length of the non-flexible fixed end.

In some embodiments, the hollow tip has a tapered shape, having an outer diameter which decreases moving along a longitudinal axis of the hollow tip in a direction from the non-flexible fixed end toward the flexible free end.

In some embodiments, the hollow tip has a flexible free end opening having a flexible free end opening inner diameter of 0.5 to 5 mm.

In some embodiments, the hollow tip further comprises a visual size indicator.

In some embodiments, the suction tube and/or the hollow tip further comprise a light guide substantially oriented along a longitudinal axis of the suction tube and/or the hollow tip and the light guide has a source end and an illumination end.

In some embodiments, the ear suctioning device comprises a light source configured to provide illumination to the source end of the light guide.

In some embodiments, the ear suctioning device comprises a hollow tip ejection mechanism which allows the hollow tip to be disconnected and removed from the suction tube without a user making contact with the hollow tip.

The present disclosure also relates to a method of providing suction to an ear canal. The method comprises placing in the ear canal the hollow tip and optionally the distal end of the ear suctioning device and applying suction.

In some embodiments, the method comprises reversibly occluding the pressure-equalizing hole to adjust a suction strength applied to the ear canal, in which the occluding is either partial occluding or total occluding.

In some embodiments, the method further comprises removing from the ear suctioning device of claim 1 the hollow tip to form a tipless ear suctioning device and a used tip and disposing of the used tip.

These and other aspects and features of non-limiting embodiments of the present disclosure will become apparent to those skilled in the art upon review of the following description of specific non-limiting embodiments of the disclosure in conjunction with the accompanying drawings.

### BRIEF DESCRIPTION OF THE DRAWINGS

A better understanding of embodiments of the present disclosure (including alternatives and/or variations thereof) may be obtained with reference to the detailed description of the embodiments along with the following drawings, in which:

FIG. 1 is a schematic sectional view of an exemplary ear and a schematic perspective view of an ear suctioning device placed within an ear canal of the ear, according to an embodiment of the present disclosure;

FIG. 2 is a longitudinal sectional view of a suction tube of the ear suctioning device, according to an embodiment of the present disclosure;

FIG. 3A illustrates an occlusion mechanism for a pressure-equalizing hole of the ear suctioning device, according to one embodiment of the present disclosure;

FIG. 3B illustrates an occlusion mechanism for the pressure-equalizing hole of the ear suctioning device, according to another embodiment of the present disclosure;

FIG. 4 is a longitudinal sectional view of a hollow tip of the ear suctioning device, according to an embodiment of the present disclosure;

FIG. 5A is a longitudinal sectional view of a hollow tip of the ear suctioning device, according to one embodiment of the present disclosure;

FIG. 5B is a longitudinal sectional view of a hollow tip of the ear suctioning device, according to another embodiment of the present disclosure;

FIG. 5C is a longitudinal sectional view of a hollow tip of the ear suctioning device, according to yet another embodiment of the present disclosure;

FIG. 6 is a schematic diagram of a tip interlock of the ear suctioning device, according to an embodiment of the present disclosure;

FIG. 7 is a schematic perspective view of the ear suctioning device showing an exemplary light guide, according to an embodiment of the present disclosure;

FIG. 8 is a schematic diagram of a hollow tip ejection mechanism of the ear suctioning device, according to an embodiment of the present disclosure; and

FIG. 9 is a flow diagram of a method of providing suction to the ear canal using the ear suctioning device, according to an embodiment of the present disclosure.

### DETAILED DESCRIPTION

In the following description, it is understood that other embodiments may be utilized, and structural and operational changes may be made without departure from the scope of the present embodiments disclosed herein.

As used herein, the terms “optional” or “optionally” means that the subsequently described event(s) can or cannot occur or the subsequently described component(s) may or may not be present (e.g., 0 wt. %).

As used herein, the term “fluidly coupled” or “fluidly connected” refer to structures, such as tubes, which are connected or coupled so as to allow a fluid (i.e. a liquid or gas) to pass between the structures. Structures said to be “fluidly coupled” or “fluidly connected” may or may not allow the fluid to leak or escape the structures. Structures which allow a fluid to pass between the structures are said to be in “fluid communication”.

According to a first aspect, an ear suctioning device is disclosed for performing ear or ear canal cleaning operation without causing any injuries to the ear canal or discomfort to patients. The ear suctioning device comprises a non-disposable suction tube having a distal segment and a proximal segment which are angled with respect to each other at about 130 to 170 degrees. A disposable hollow tip is reversibly and fluidly connected to the distal segment. The proximal segment is configured to attach to a vacuum or low pressure line. A pressure-equalizing hole is disposed on the proximal segment to control air pressure or suction strength within an interior volume of the suction tube and thereby to extract wax, debris and other foreign objects from the ear canal.

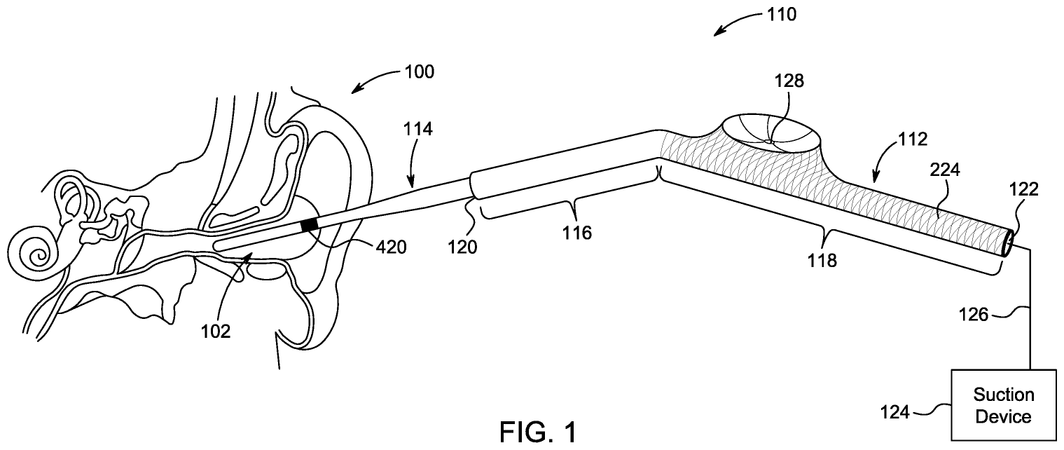

FIG. 1 illustrates a schematic sectional view of an exemplary ear 100 of a patient requiring cleaning operation in an ear canal 102. As shown in FIG. 1, a schematic perspective view of an ear suctioning device 110 is illustrated, according to an embodiment of the present disclosure. At least a portion of the ear suctioning device 110 is shown substantially aligned with and placed within the ear canal 102. The ear suctioning device 110 is used to perform the cleaning operation in the ear canal 102 of the patient. Specifically, the ear suctioning device 110 may be used to clean wax, discharges, fluids, and/or other debris from the ear canal 102. The ear suctioning device 110 comprises a suction tube 112 and a hollow tip 114 reversibly connected to the suction tube 112. The suction tube 112 comprises a distal segment 116 and a proximal segment 118. The phrase ‘distal segment’ may be understood as the segment that is located away from or farther from an operator of the ear suctioning device 110 while the operator is performing the cleaning operation. The phrase ‘proximal segment’ may be understood as the segment that is located nearer the operator while the operator is performing the cleaning operation. The distal segment 116 and the proximal segment 118 are angled with respect to each other at about 130 to 170 degrees. The suction tube 112 is hollow, having an interior volume 130 defined by an interior surface of the suction tube 112.

In general, the suction tube may be constructed of any material with sufficient stiffness so as to not collapse when suction is turned on or applied. In some embodiments, the suction tube is constructed of or an interior surface of the suction tube is lined with a non-porous material. An interior surface of the suction tube may come into contact with material removed from a patient's ear canal. Such material may represent a biohazard or other type of hazard to the patient, operator, or other person who may come into contact with the device. It would be advantageous to completely remove such material from the suction tube during cleaning so as to minimize the risks or hazards posed by the material. Porous materials comprise voids or pores in which the material may be trapped and therefore not removed during cleaning. In some embodiments, the suction tube 112 comprises a biocompatible metal. The term “biocompatible metal” refers to a non-toxic and non-irritating metallic material which is safe to the human body. Examples of biocompatible metals include stainless steels and stainless steel alloys, cobalt-chromium alloys, and titanium alloys. Biocompatible stainless steels may be those which are compliant with specific guidelines published by standards organizations known to one or ordinary skill in the art such as International Organization for Standards (ISO) 5832-1, ISO 10993-6, ISO 10993-10, ISO 10993-11, and ASTM F-138. Examples of biocompatible stainless steels include 316 stainless steels, 422 stainless steels, 440 stainless steels. Biocompatible titanium alloys may be those which are compliant with specific guidelines published by standards organizations known to one or ordinary skill in the art such as ASTM F136, ASTM F67, and ISO 5832-3. Biocompatible cobalt-chromium alloys may be those which are compliant with specific guidelines published by standards organizations known to one of ordinary skill in the art such as ASTM F75. Biocompatible cobalt-chromium alloys may comprise other metals, such as molybdenum and/or nickel. In some embodiments, the materials used to make the suction tube 112 may be a stainless-steel alloy, a cobalt-chromium alloy, a titanium alloy, or any other metal alloy known to one of ordinary skill in the art.

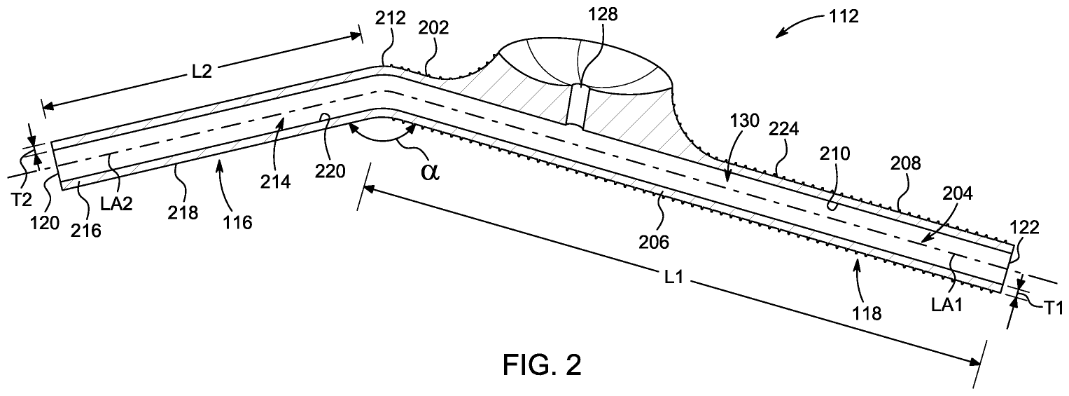

The suction tube 112 further comprises a distal end 120 defined at the distal segment 116 and a proximal end 122 defined at the proximal segment 118. The distal end 120 of the suction tube 112 is configured to reversibly connect to the hollow tip 114 as such the hollow tip 114 is fluidly connected to the suction tube 112. The distal end 120 of the suction tube 112 is open, allowing a fluid to pass into or out of the suction tube 112. The proximal end 122 of the suction tube 112 is configured to couple with a suction generation device 124 through a vacuum or low pressure line 126, which is hereinafter referred to as ‘the vacuum line 126’. Similarly, the proximal end 122 of the suction tube 112 is open, allowing a fluid to pass into or out of the suction tube 112. The suction tube 112 further comprises a pressure-equalizing hole 128 disposed on the proximal segment 118. The pressure-equalizing hole 128 is in fluid communication with the interior volume 130 (shown in FIG. 2) of the suction tube 112.

Referring to FIG. 2, a longitudinal sectional view of the suction tube 112 is illustrated, according to an embodiment of the present disclosure. The suction tube 112 comprises the proximal segment 118 and the distal segment 116. The proximal segment 118 includes a first end, otherwise referred to as ‘the proximal end 122’, and a second end 202 distal to the proximal end 122. The proximal end 122 of the proximal segment 118 is configured to couple with the suction generation device 124 though the vacuum line 126. Particularly, the proximal end 122 is fluidly coupled with the vacuum line 126. In some embodiments, the vacuum line 126 may be a flexible tube or hose configured to reversibly couple with the proximal end 122 of the suction tube 112. In some embodiments, the vacuum line 126 is a flexible pipe. In such embodiments the flexible pipe may be any suitable length to allow operation of the ear suctioning device without impeding movement or positioning of the ear suctioning device. In some embodiments, the vacuum line 126 may be made of plastic, polymer, stainless steel or any other suitable material known to one of ordinary skill in the art.

The suction generation device 124 may be defined as a vacuum creating device used for creating vacuum or an air pressure lower than ambient air pressure within the suction tube 112. The vacuum or low pressure within the suction tube 112 may cause an inflow of air and/or other material through the distal end 120, the hollow tip 114 (if attached), and/or the pressure-equalizing hole 128. In general, the suction generation device 124 may be any suitable device for achieving suction known to one of ordinary skill in the art. In some embodiments, the suction generation device 124 may be a portable or a handheld device adapted to couple with the proximal end 122 of the suction tube 112. In such embodiments, the proximal end 122 may be detachably coupled with the vacuum line 126. In alternative embodiments, the proximal end 122 may be permanently coupled with the vacuum line 126 associated with the suction generation device 124. In such embodiments, the ear suctioning device 110 and the suction generation device 124 may be formed as a single unit. In some embodiments, the suction tube 112 may be configured to fluidly couple with a suction device that may be used for various other domestic or commercial purposes. In such embodiments, the proximal end 122 may be provided with a universal coupling mechanism that may be configured to couple with a vacuum line of any size or shape known to one of ordinary skill in the art. In some embodiments, the proximal end 122 may be fluidly coupled with the vacuum line 126 with a coupling mechanism including, but not limited to, a threadable mechanism, a press-fit or interference fit mechanism, a snap-fit mechanism, or any other coupling mechanism known to one of ordinary skill in the art. In some embodiments, the proximal end 122 may be provided with bumps, barbs, ridges, or other structure configured to facilitate secure coupling with the vacuum line 126. In some embodiments, the vacuum line 126 is secured by an external clamp placed around an exterior surface of the vacuum line.

The proximal segment 118 has a length ‘L1’ defined between the proximal end 122 and the second proximal segment end 202. In some embodiments, the length ‘L1’ of the proximal segment 118 is 12 to 5 cm, preferably 11 to 6 cm, preferably 10 to 7 cm, preferably 9 to 8 cm. In some embodiments, the proximal segment 118 has a circular cross-section defining an outer diameter. In some embodiments, the outer diameter of the proximal segment 118 is 10 to 6 mm, preferably 9 to 7 mm, preferably 7 mm. In some embodiments, cross-sectional shape of the proximal segment 118 may be an oval, an elliptical, a polygon, or any other shape known to one of ordinary skill in the art. In some embodiments, cross-sectional area of the proximal segment 118 may be same throughout the length ‘L1’ thereof. In alternative embodiments, the cross-sectional area of the proximal segment 118 may progressively decrease from the proximal end 122 to the second proximal segment end 202 or from the second proximal segment end 202 to the proximal end 122. The proximal segment 118 may be made of materials including, but not limited to, metal, plastic, polymers, nontoxic hard material or any other biocompatible metal known to one of ordinary skill in the art as described above. The proximal segment 118 further comprises a first lumen 204 defined along a first longitudinal axis ‘LA1’ thereof. The first lumen 204 is further defined throughout the length ‘L1’ of the proximal segment 118. In some embodiments, the first lumen is an interior surface of the proximal segment 118. In some embodiments, the first lumen 204 has a circular cross-section defining an inner diameter. In some embodiments, the inner diameter is 8 to 4 mm, preferably 7 to 5 mm, preferably 5 mm. In some embodiments, cross-section of the first lumen 204 may have an oval shape, elliptical shape, polygon or any other shape known to one of ordinary skill in the art. In some embodiments, the first lumen 204 has a cross-sectional shape that is the same as the cross-sectional shape of the proximal segment 118. In alternative embodiments, the first lumen 204 and the proximal segment 118 have different cross-sectional shape. The proximal segment 118 further comprises a wall 206 having a thickness ‘T1’ defined between an outer surface 208 and an inner surface 210 defined by the first lumen 204. In some embodiments, the thickness ‘T1’ of the wall 206 of the proximal segment 118 may be of 2 to 0.5 mm, preferably 1 mm.

The distal segment 116 comprises a first end, otherwise referred to as ‘the distal end 120, and a second distal segment end 212. The distal end 120 of the distal segment 116 is configured to reversibly and fluidly connect with the hollow tip 114. The distal segment 116 has a length ‘L2’ defined between the distal end 120 and the second distal segment end 212. In some embodiments, the length ‘L2’ of the distal segment 116 is 5 to 12 cm, preferably 6 to 11 cm, preferably 7 to 10 cm, preferably 8 to 9 cm. In some embodiments, the distal segment 116 has a circular cross-section defining an outer diameter. In some embodiments, the outer diameter of the distal segment 116 is 10 to 6 mm, preferably 9 to 7 mm, preferably 7 mm. In some embodiments, cross-sectional shape of the distal segment 116 may be an oval, an elliptical, a polygon, or any other shape known to one of ordinary skill in the art. In some embodiments, cross-sectional area of the distal segment 116 may be same throughout the length ‘L2’ thereof. In alternative embodiments, the cross-sectional area of the distal segment 116 may progressively decrease from the distal end 120 to the second distal segment end 212 or from the second distal segment end 212 to the distal end 120. The distal segment 116 may be made of materials including, but not limited to, metal, plastic, polymers, nontoxic hard material or any other biocompatible metal known to one of ordinary skill in the art as described above. The distal segment 116 further comprises a second lumen 214 defined along a second longitudinal axis ‘LA2’ thereof. The second lumen 214 is further defined throughout the length ‘L2’ of the distal segment 116. In some embodiments, the second lumen 214 has a circular cross-section defining an inner diameter. In some embodiments, the inner diameter of the second lumen 214 is 8 to 4 mm, preferably 7 to 5 mm, preferably 5 mm. In some embodiments, the cross-section of the second lumen 214 may have an oval shape, elliptical shape, polygon shape, or any other shape known to one of ordinary skill in the art. In some embodiments, the second lumen 214 has a cross-sectional shape that is the same as the cross-sectional shape of the distal segment 116. In alternative embodiments, the second lumen 214 and the distal segment 116 have different cross-sectional shapes. The distal segment 116 further includes a wall 216 having a thickness ‘T2’ defined between an outer surface 218 and an inner surface 220 defined by the second lumen 214. In some embodiments, the thickness ‘T2’ of the wall 216 of the distal segment 116 may be of 2 to 0.5 mm, preferably 1 mm. In FIG. 2, an embodiment of the device is depicted where the length ‘L1’ of the proximal segment 118 is 10 cm and the length ‘L2’ of the distal segment 116 is 5 cm.

The proximal segment 118 and the distal segment 116 are angled with respect to each other at an angle ‘α’. In some embodiments, the angle ‘α’ may be in a range of about 130 to 170 degrees, preferably 135 to 165 degrees, preferably 140 to 160 degrees, preferably 142.5 to 157.5 degrees, preferably 145 to 155 degrees, preferably 147.5 to 152.5 degrees, preferably 150 degrees. Particularly, the first longitudinal axis ‘LA1’ of the proximal segment 118 and the second longitudinal axis ‘LA2’ of the distal segment 116 are angled at about 130 to 170 degrees, preferably 135 to 165 degrees, preferably 140 to 160 degrees, preferably 142.5 to 157.5 degrees, preferably 145 to 155 degrees, preferably 147.5 to 152.5 degrees, preferably 150 degrees. The angled design of the suction tube 112 makes the hand that holds the ear suctioning device 110 does not interfere with physician's vision field of the ear 100 to be cleaned as the ear canal 102 is narrow and any medical intervention needs special skills and clear vision field.

In some embodiments, the proximal segment 118 and the distal segment 116 may be individual or distinct components which may be either reversibly coupled to each other or permanently connected to each other. In such embodiments, the proximal segment 118 and the distal segment 116 may be manufactured separately. In some embodiments, the second proximal segment end 202 of the proximal segment 118 and the second distal segment end 212 of the distal segment 116 are detachably or permanently connected to each other. In case of detachable coupling, the proximal segment 118 and the distal segment 116 may be connected using fasteners such as nuts and bolts, press-fit, snap-fit, or any other detachable coupling mechanism known to one of ordinary skill in the art. In case of permanent coupling, the proximal segment 118 and the distal segment 116 may be connected using an adhesive or any other permanent coupling mechanism known to one of ordinary skill in the art.

In some embodiments, the proximal segment 118 and the distal segment 116 are integrated to form as a single component or are constructed as a single component as shown in FIG. 2. In such embodiments, the proximal segment 118 and the distal segment 116 may be manufactured together as a single component. The suction tube 112 has a total length defined between the proximal end 122 and the distal end 120. In some embodiments, the total length of the suction tube 112 may be measured along a horizontal plane defined by the proximal end 122 and the distal end 120. In such embodiments, the total length of the suction tube 112 may be about 12 to 18 cm, preferably 13 to 17 cm, preferably 14 to 16 cm, preferably 15 cm. In some embodiments, the total length of the suction tube 112 may be measured along the length ‘L1’ of the proximal segment 118 and the length ‘L2’ of the distal segment 116. In other words, the total length of the suction tube 112 may be sum of the length ‘L1’ of the proximal segment 118 and the length ‘L2’ of the distal segment 116. The first lumen 204 of the proximal segment 118 and the second lumen 214 of the distal segment 116 together form a passage in the suction tube 112. The passage may be further defined as the interior volume 130 of the suction tube 112. The interior volume 130 of the suction tube 112 is configured to be in fluid communication with the vacuum line 126 and the hollow tip 114. As such, the suction generation device 124 coupled to the proximal end 122 of the suction tube 112 is configured to create a vacuum within the interior volume 130 of the suction tube 112 during the ear cleaning operation.

The ear suctioning device 110 further comprises the pressure-equalizing hole 128 disposed in the proximal segment 118 of the suction tube 112. The pressure-equalizing hole 128 is configured to control air pressure within the interior volume 130 of the suction tube 112. To accomplish this, the pressure-equalizing hole 128 is in fluid communication with the interior volume 130. The pressure-equalizing hole 128 may be fully opened, partially opened or closed to control or adjust vacuum, suction strength, or air pressure within the interior volume 130 during the ear cleaning operation. The pressure-equalizing hole 128 is disposed in the proximal segment 118 adjacent to the second proximal segment end 202 thereof. In some embodiments, the pressure-equalizing hole 128 may be disposed anywhere between the proximal end 122 and the second proximal segment end 202 of the proximal segment 118 in such a way to allow the operator of the ear suctioning device 110 to conveniently operate the pressure-equalizing hole 128. In some embodiments, a central axis defined by the pressure-equalizing hole 128 may be perpendicular to the first longitudinal axis ‘LA1’ of the proximal segment 118. In alternative embodiments, the central axis of the pressure-equalizing hole 128 may be inclined at an angle with respect to the first longitudinal axis ‘LA1’ of the proximal segment 118. In some embodiments, a diameter of the pressure-equalizing hole 128 may be same throughout the thickness ‘T1’ of the wall 206 of the proximal segment 118 to fluidly communicate with the first lumen 204 of the proximal segment 116 and thereby the interior volume 130 of the suction tube 112. In alternative embodiments, the pressure-equalizing hole 128 may have a tapering cross-section along the thickness ‘T1’ of the wall 206 of the proximal segment 118. The pressure equalizing hole 128 has an outer opening located at or on the outer surface 208 of the proximal segment 118 and an inner opening located at or on the first lumen 204 of the proximal segment 118. In some embodiments, the outer opening has an outer opening diameter that is the same as an inner opening diameter of the inner opening. In alternative embodiments, the outer opening diameter is larger or smaller than the inner opening diameter.

In general, the suction tube 112 may be manufactured by any suitable method known to one of ordinary skill in the art. Examples of such methods include, but are not limited to, injection molding, extrusion, forming, rolling, drawing, and milling. In some embodiments, the proximal segment 118 and/or the distal segment 116 are seamless. In alternative embodiments, the proximal segment 118 and/or the distal segment 116 are not seamless. In this context, “seamless” refers to a tube or other similar structure which does not have a seam resulting from welding or other metal joining process. Seamless tubing is distinct from welded tubing, which has a seam which results from welding or other joining process. Such welded tubing is typically formed by rolling or otherwise forming a tubular shape having an interior volume out of a shape which does not enclose an interior volume, typically a flat sheet. Edges of the flat sheet are secured, typically by welding, to join the edges and close the tube to enclose the interior volume. This joining process leaves a distinct seam where the edges were joined. In contrast, seamless tubing is typically formed from a molten material via molding, extruding, or other similar technique or from a solid material via drawing, milling, or other similar technique. In some embodiments, the proximal segment 118 and the distal segment 116 are manufactured via the same technique. In some embodiments, the proximal segment 118 and the distal segment 116 are not manufactured via the same technique.

In some embodiments, the ear suctioning device 110 further comprises an ergonomic grip 224 located on or attached to the proximal segment 118 of the suction tube 112. In some embodiments, the ergonomic grip 224 is provided on the outer surface 208 of the proximal segment 118. In some embodiments, the ergonomic grip 224 extends along the entirety of the length ‘L1’ of the proximal segment 118. In alternative embodiments, the ergonomic grip 224 does not extend along the entirety of the length ‘L1’ of the proximal segment 118. In such embodiments, the ergonomic grip 224 may be formed on the outer surface 208 of the proximal segment 118 while the proximal segment 118 is being manufactured. In some embodiments, the ergonomic grip 224 may be defined on the proximal segment 118 using manufacturing process such as knurling, or any other process known to one of ordinary skill in the art to provide handgrip on the proximal segment 118.

In some embodiments, the ergonomic grip 224 is reversibly attached to the proximal segment 118. In such embodiments, the ergonomic grip 224 is manufactured separately and attached to the proximal segment 118. In some embodiments, the ergonomic grip 224 is attached to the outer surface 208 of the proximal segment 118 using an adhesive or any other attaching mechanisms known to one of ordinary skill in the art. In some embodiments, the ergonomic grip 224 is attached to the outer surface 208 of the proximal segment 118 or the proximal end 122 of the proximal segment 118 using fastening mechanisms such as bolts and nuts, snap-fit, press-fit, or any other fastening mechanisms known to one of ordinary skill in the art. In some embodiments, the ergonomic grip 224 is made of a silicone material, a memory foam, or other elastomeric material known to one of ordinary skill in the art. In some embodiments, the ergonomic grip 224 may be formed by embossing finger profiles on the proximal segment 118 such that fingers of the operator may firmly wrap around the proximal segment 118 to comfortably hold the ear suctioning device 110. In some embodiments, the ergonomic grip 224 may be located on or attached to both the proximal segment 118 and the distal segment 116. In some embodiments, the ergonomic grip 224 is located on or attached to the distal segment 116 in such a way that the ergonomic grip 224 and the proximal segment 118 together allow the operator to comfortably hold the ear suctioning device 110 during the ear cleaning operation. In some embodiments, at least a portion of the ergonomic grip 224 encompasses an entirety of a circumference of the proximal segment 118. In alternative embodiments, no portion of the ergonomic grip 224 encompasses an entirety of the circumference of the proximal segment 118. In general, the ergonomic grip 224 may be of any suitable shape known to one of ordinary skill in the art. Such a shape may have depressions or projections configured to conform to a user's hand, finger, and/or portion thereof. In some embodiments, the ergonomic grip 224 comprises bumps, ridges, or other surface texture known to one of ordinary skill in the art. Such surface texture may serve to increase friction or otherwise serve to secure the ear suctioning device 110 in a user's grip. In some embodiments, the ergonomic grip 224 comprises cushioning. Such cushioning may be in the form of pads, rings, tubes, or other structure. In some embodiments, the cushioning is compressible. Such cushioning may serve to increase the comfort of a user holding the ear suctioning device 110. Examples of ergonomic grips may be found in U.S. Pat. Nos. 6,779,937B1, 6,390,818B2, 5,979,015A, 8,850,662B2, and 4,785,495A.

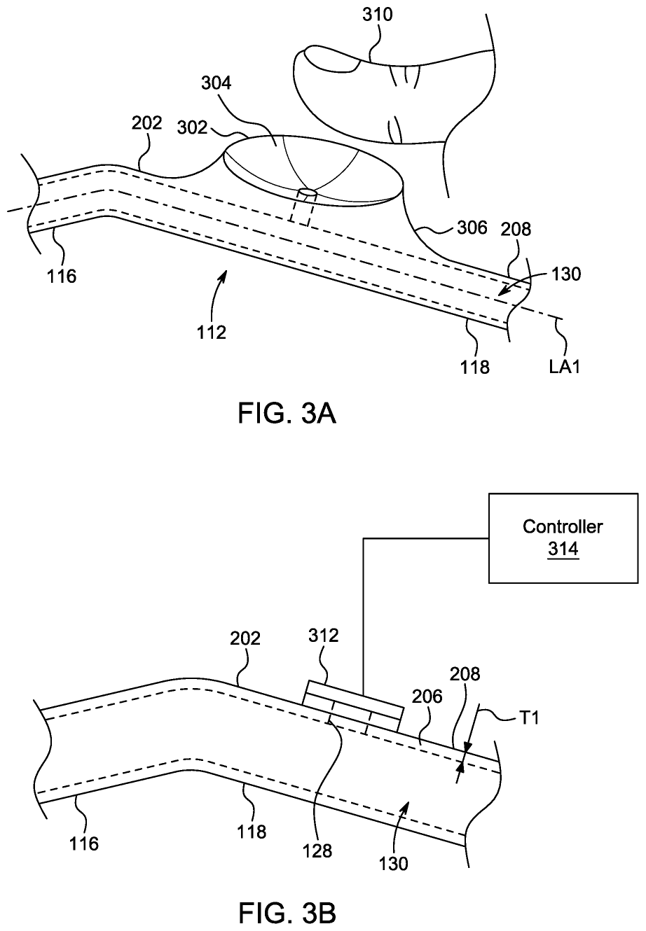

Referring to FIG. 3A, an occlusion mechanism for the pressure-equalizing hole 128 is illustrated, according to an embodiment of the present disclosure. As shown in FIG. 3A, the occlusion mechanism is illustrated as a manual occlusion mechanism. In some embodiments, the pressure-equalizing hole 128 is located in a finger cup 302. In some embodiments, the finger cup 302 has a substantially hemiovoid shape. In some embodiments, the finger cup 302 is disposed adjacent the second proximal segment end 202 of the proximal segment 118. In some embodiments, the finger cup 302, along with the pressure-equalizing hole 128, may be located anywhere along the length ‘L1’ of the proximal segment 118. In some embodiments, the finger cup 302 comprises a curved inner surface 304 and a curved outer surface 306 and extends radially away from the outer surface 208 of the proximal segment 118. In some embodiments, the finger cup 302 may extend radially and perpendicularly with respect to the first longitudinal axis ‘LA1’ of the proximal segment 118. In alternative embodiments, the finger cup 302 may extend radially and at an inclination with respect to the first longitudinal axis ‘LA1’ of the proximal segment 118. In some embodiments, the curved inner surface 304 may be formed as the hemiovoid shape. The hemiovoid shape may be defined as a three-dimensional (3D) shape of a half of egg-shaped structure. In some embodiments, the curved inner surface 304 of the finger cup 302 may have a substantially hemiellipsoid shape, a hemispherical shape, or any other shape known to one of ordinary skill in the art. In some embodiments, the finger cup 302 is part of the ergonomic grip 224. In some embodiments, the finger cup 302 is formed from the proximal segment 118. In such embodiments, the ergonomic grip 224 may encompass a portion of the finger cup 302.

The finger cup 302 having the hemiovoid shape is configured in such a way that the pressure-equalizing hole 128 is capable of being partially or totally occluded by a finger or a thumb 310 placed in the finger cup 302. In some embodiments, the curved inner surface 304 may be defined in such a way to conform with a curved shape of the finger or the thumb 310. In such embodiments, the finger or the thumb 310 of the operator may be firmly seated within the finger cup 302 having the hemiovoid shape. Further, the pressure-equalizing hole 128 defined within the finger cup 302 is fluidly communicated with the interior volume 130 of the suction tube 112. As such, during the ear cleaning operation, the finger or the thumb 310 of the operator may be used to partially or totally occlude the pressure-equalizing hole 128 and thereby control the vacuum pressure within the interior volume 130 of the suction tube 112. In some embodiments, the operator may partially or totally occlude the pressure-equalizing hole 128 based on the pressure within the interior volume 130 of the suction tube 112 sensed by the skin of the finger or the thumb 310 of the operator. In some embodiments, the finger cup 302 has bumps, ridges, projections, or other surface texture. Such surface texture may increase the comfort of a user or increase the security contact between a finger and the finger cup 302. In some embodiments, the surface texture comprises an elastomeric material as described above. In alternative embodiments, the finger cup 302 is substantially free of surface texture. Such surface texture may interfere with secure partial or total occlusion of the pressure-equalizing hole 128.

Referring to FIG. 3B, an occlusion mechanism for the pressure-equalizing hole 128 is illustrated, according to an embodiment of the present disclosure. As shown in FIG. 3B, the occlusion mechanism is illustrated as a controllable pressure-equalizing hole occlusion mechanism 312, otherwise referred to as ‘the controllable occlusion mechanism 312’. A schematic representation of the controllable occlusion mechanism 312 is shown in FIG. 3B for mere purpose of illustration and not to limit the scope of the present disclosure as claimed. The pressure-equalizing hole 128 is defined throughout the thickness ‘T1’ of the wall 206 of the proximal segment 118 and the controllable occlusion mechanism 312 may be disposed on the proximal segment 118 to partially or totally occlude the pressure-equalizing hole 128. In some embodiments, the controllable occlusion mechanism 312 may include, but is not limited to, a cap, a cover, a lid, an iris, or any other device known to one of ordinary skill in the art to selectively close or open the pressure-equalizing hole 128. In some embodiments, the controllable occlusion mechanism 312 is a radial iris mechanism. In some embodiments, the controllable occlusion mechanism 312 is slidably, pivotally, rotatably or movably connected to or disposed on the proximal segment 118 to partially to totally occlude the pressure-equalizing hole 128. In some embodiments, the controllable occlusion mechanism 312 is connected to or disposed on the outer surface 208 of the proximal segment 118. In some embodiments, the controllable occlusion mechanism 312 is operated by the operator. In such embodiments, the controllable occlusion mechanism 312 is operated based on an input indicative of the vacuum pressure within the interior volume 130 of the suction tube 112. The input indicative of the vacuum pressure within the interior volume 130 of the suction tube 112 may be displayed in a display device (not shown), such that the operator may control the movement of the controllable occlusion mechanism 312 based on the value of vacuum pressure displayed in the display device.

In some embodiments, a pressure detection unit (not shown) may be disposed within the interior volume 130 or the proximal end 122 of the suction tube 112. The pressure detection unit may be configured to generate the input indicative of the vacuum pressure within the interior volume 130 of the suction tube 112. The pressure detection unit may be further communicated with the display device to display the value indicative of the vacuum pressure. In some embodiments, the display device may be disposed on the proximal segment 118 of the suction tube 112. In such embodiments, the display device may be a small LED display, or any compact display known to one of ordinary skill in the art to attach with the proximal segment 118 of the suction tube 112. In some embodiments, the display device may be integral with the proximal segment 118 of the suction tube 112. In alternative embodiments, the display device may be an external device, used for other applications, separately communicated with the pressure detection unit of the ear suctioning device 110. In such embodiments, the display device may be a TV screen, monitor or any other display device that may be used for various other domestic or commercial purposes.

In some embodiments, the ear suctioning device 110 may include a controller 314 in communication with the controllable occlusion mechanism 312. In some embodiments, the controller 314 is disposed on or attached to the proximal segment 118 of the suction tube 112. In alternative embodiments, the controller is not attached to or disposed on the proximal segment 118. In such embodiments, the controller may be a part of, attached to, or otherwise integrated with the suction generation device 124. In some embodiments, the controller 314 may be in a wired communication or a wireless communication with a sensor (not shown) located on the proximal segment 118. The controller 314 may be configured to receive a signal indicative of the vacuum pressure within the interior volume 130 of the suction tube 112 and determine a value of the vacuum pressure based on the signal received from the sensor. The controller 314 may be further communicated with the controllable occlusion mechanism 312 and configured to actuate the controllable occlusion mechanism 312 to partially or totally occlude the pressure-equalizing hole 128 based on the determined vacuum pressure. In some embodiments, one or more target vacuum pressures may be preset by the operator in the controller 314. The target vacuum pressure(s) may be defined based on various factors including, but not limited to, age of the patient and diagnostic condition of the ear canal 102 of the patient. The target vacuum pressure(s) may be stored in a memory of the controller 314. During the ear cleaning operation, the operator may preset the target vacuum pressure(s) in the controller 314. The controller 314 in communication with the senor may further determine the actual vacuum pressure within the interior volume 130 of the suction tube 112. The actual vacuum pressure may be further compared with the target vacuum pressure and actuate the controllable occlusion mechanism 312 based on a difference value between the target vacuum pressure and the actual vacuum pressure. In some embodiments, the controller 314 may be an external device, used for other applications, separately communicated with the sensor and the controllable occlusion mechanism 312 and configured to actuate the controllable occlusion mechanism 312 based on the difference value between the target vacuum pressure and the actual vacuum pressure. In some embodiments, the controller 314 may operate the controllable occlusion mechanism 312 based on user input. In such embodiments, the user input may be provided by any suitable method known to one of ordinary skill in the art. Examples of such input methods include, but are not limited to buttons, pressure sensors, triggers, sliders, dials, and levers.

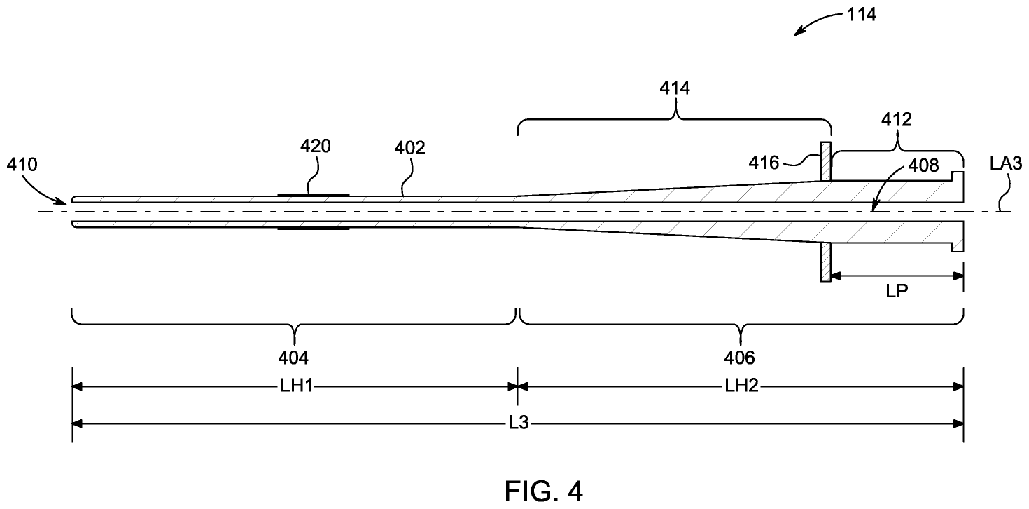

Referring to FIG. 4, a longitudinal sectional view of the hollow tip 114 of the ear suctioning device 110 is illustrated, according to an embodiment of the present disclosure. The hollow tip 114 is reversibly connected to the distal segment 116 of the suction tube 112. Further, the hollow tip 114 is fluidly connected to the distal end 120 of the suction tube 112. In some embodiments, the hollow tip 114 includes an elongated hollow body 402 having a free end 404 and a fixed end 406. In some embodiments, the elongated hollow body 402, the free end 404, and/or the fixed end 406 are flexible. In some embodiments, the fixed end 406 is non-flexible. The elongated hollow body 402 may have a length ‘L3’ defined by the free end 404 and the fixed end 406 along a longitudinal axis ‘LA3’ thereof. The free end 404 may have a first length ‘LH1’ and the fixed end 406 may have a second length ‘LH2’. In some embodiments, the first length ‘LH1’ of the free end 404 may be defined based on various factors including, but not limited to, an average length of the ear canal 102 of a child and an adult. Such that, when the hollow tip 114 is inserted into the ear canal 102 of the patient, any incident of injury or inconvenience to the patient due to the fixed end 406 may be avoided. Similarly, the second length ‘LH2’ of the fixed end 406 may be defined based on various factors including, but not limited to, coupling arrangement between the hollow tip 114 and the distal segment 116 of the suction tube 112 and cross-sectional area of the hollow tip 114 and the distal segment 116. As such, the first length ‘LH1’ of the free end 404 and the second length ‘LH2’ of the fixed end 406 together define the length ‘L3’ for the hollow tip 114. In other words, sum of the first length ‘LH1’ of the free end 404 and the second length ‘LH2’ of the fixed end 406 is equal to the length ‘L3’ of the hollow tip 114. In some embodiments, each of the first length ‘LH1’ and the second length ‘LH2’ of the free end 404 and the fixed end 406, respectively, may be of about 20 mm to 50 mm, preferably 22.5 to 45 mm, preferably 25 to 40 mm, preferably 27.5 to 35 mm, preferably 30 mm. As shown in FIG. 4, the first length ‘LH1’ of the free end 404 is 30 mm and the second length ‘LH2’ of the fixed end 406 is 30 mm, hence the length ‘L3’ of the hollow tip 114 is 60 mm. In some embodiments, the first length ‘LH1’ of the free end 404 and the second length ‘LH2’ of the fixed end 406 are equal to each other. In alternative embodiments, the first length ‘LH1’ of the free end 404 and the second length ‘LH2’ of the fixed end 406 are not equal to each other.

The free end 404 is configured to be inserted into the ear canal 102 of the patient for performing the cleaning operation of the ear 100. The fixed end 406 is configured to connect to the distal segment 116 of the suction tube 112. Particularly, the fixed end 406 is configured to reversibly connect to the distal end 120 of the suction tube 112. In general, the hollow tip 114 may be made of any suitable body-safe material known to one of ordinary skill in the art. In this context, “body-safe material” refers to a material which is non-toxic, non-irritating, or otherwise generally recognized as safe for contact with body tissues such as the ear or ear canal. In this context, a synonym for “body-safe” is “biocompatible”. In some embodiments, the body-safe material is a body-safe polymer. Examples of body-safe polymers include polyethylene, polypropylene, ethylene vinyl acetate co-polymer, acrylonitrile butadiene styrene (ABS), silicone, poly(2-methoxyethyl acrylate) (PMEA), poly(tetrahydrofurfuryl acrylate) (PTHFA), polylactic-co-glycolic acid, poly(ε-caprolactone) (PCL), poly-lactic acid, and poly(3-hydroxybutyrate-co-3-hydroxyvalerate) (PHBV). In some embodiments, the body-safe material is a body-safe plastic. In some embodiments, the body-safe material is a body-safe metal. In some embodiments where the free end 404 is flexible, the free end 404 comprises an elastomeric material. In some embodiments, the free end 404 comprises a silicone. In some embodiments, the free end 404 may be made of any suitable non-toxic flexible material known to one of ordinary skill in the art. In some embodiments, the fixed end 406 is comprises an elastomeric material. In some embodiments, the fixed end 406 is constructed of the same material or materials as the free end 404. In some embodiments, the fixed end 406 comprises a body-safe plastic. In some embodiments, the body-safe plastic is a rigid plastic. In alternative embodiments, the body-safe plastic is a non-rigid plastic. In general, the hollow tip 114 should be constructed such that the hollow tip does not collapse as a result of the vacuum, suction, or low air pressure generated by the suction generation device.

In some embodiments, the hollow tip 114 is configured to be disposable. In preferred embodiments, the hollow tip 114 is disposed of after a single use. In such embodiments, a single use may refer to use that involves cleaning of or contact with a single ear or alternatively both ears on a single person. Preferably, the hollow tip 114 is constructed of body-safe materials which facilitate disposal. Such materials may be biodegradable or non-biodegradable. Such materials may be capable of being incinerated. In some embodiments, the hollow tip 114 is provided separate from the other components of the ear suctioning device. In such embodiments, the hollow tip 114 may be provided in packaging which is configured to keep the hollow tip 114 sterile. Such packaging may further allow the hollow tip 114 to be sterilized. The hollow tip 114 being disposable may be advantageous for preventing infection, disease spread, or other disadvantageous condition.

In some embodiments, the elongated hollow body 402 of the hollow tip 114 comprises a tip lumen 408 configured to align with the second lumen 214 of the distal segment 116 when the hollow tip 114 is engaged with the suction tube 112. The tip lumen 408 of the hollow tip 114 and the interior volume 130 of the suction tube 112 may be aligned to each other when the hollow tip 114 is engaged with the suction tube 112 to allow passage of wax, fluid, or debris therethrough during the cleaning operation of the ear canal 102. In some embodiments, cross-sectional area of the tip lumen 408 may be constant throughout the length ‘L3’ of the hollow tip 114. As such, the hollow tip 114 has a free end opening 410 having a free end opening inner diameter of 0.5 to 5 mm, preferably 1.0 to 4.5 mm, preferably 1.5 to 4 mm. Particularly, the flexible free end opening 410 may be defined by the tip lumen 408. In some embodiments, the diameter of the tip lumen 408 of the hollow tip 114 is about 0.5 to 5 mm, preferably 1.0 to 4.5 mm, preferably 1.5 to 4 mm. In some embodiments, the diameter of the tip lumen 408 of the hollow tip 114 is about 0.5 to 1.75 mm, preferably 0.75 to 1.60 mm, preferably 1.50 mm. In some embodiments, the diameter of the tip lumen 408 of the hollow tip 114 is greater than 1.75 to about 2.5 mm, preferably 1.9 to 2.25 mm, preferably 2 mm. In some embodiments, the diameter of the tip lumen 408 of the hollow tip 114 is greater than 2.5 to about 3.5, preferably 2.75 to 3.25, preferably 2.9 to 3.1 mm, preferably 3.0 mm. In some embodiments, the diameter of the tip lumen 408 of the hollow tip 114 is greater than 3.5 to 4.5 mm, preferably 3.75 to 4.25 mm, preferably 3.9 to 4.1 mm, preferably 4.0 mm. In some embodiments, a collection of hollow tips having different diameters of the tip lumen 408 are provided for use with the suction tube. In some embodiments, the diameter of the tip lumen 408 may serve as the “size” of the hollow tip. In some embodiments, the hollow tips having different diameters of the tip lumen 408 may be referred to as different sized tips. In some embodiments, the free end 404 has an outer diameter which is constant throughout the first length ‘LH1’ thereof. In some embodiments, the outer diameter of the free end 404 may be about 1.5 to 7.5 mm, preferably 2 to 7 mm, preferably 2.5 to 6 mm, preferably 3 to 5.5 mm, preferably 4 to 5 m throughout the first length ‘LH1’ thereof. In some embodiments, the free end 404 has an outer diameter which is not constant throughout the first length ‘LH1’ thereof. In such embodiments, the outer diameter of the free end 404 changes smoothly or tapers from an initial outer diameter to a final outer diameter throughout the first length ‘LH1’ thereof. As shown in FIG. 4, the outer diameter of the flexible free end 404 is 2.5 mm and the flexible free end opening inner diameter or the diameter of the lumen 408 is 1.5 mm.

In some embodiments, the fixed end 406 comprises a projection portion 412 which is configured to fit securely into the distal segment 116 of the suction tube 112. Particularly, the projection portion 412 of the fixed end 406 is configured to reversibly connect with the distal end 120 of the suction tube 112. In some embodiments, the projection portion 412 has a projection portion length ‘LP’ which is about 25 to 75% of a total length, which is otherwise referred to as ‘the second length LH2’, of the non-flexible fixed end 406. In some embodiments, the projection portion length ‘LP’ may be 30 to 70%, preferably 35 to 65%, preferably 40 to 60%, preferably 45 to 55%, of the second length ‘LH2’ of the non-flexible fixed end 406. As shown in the exemplary embodiment depicted in FIG. 4, the length ‘L3’ of the hollow tip 114 is 60 mm, in which the second length ‘LH2’ of the non-flexible fixed end 406 is 30 mm and the projection portion length ‘LP’ is 10 mm. In some embodiments, the projection portion 412 may have an outer diameter defined based on the inner diameter of the second lumen 214 such that the projection portion 412 is slidably received within the second lumen 214 at the distal end 120 of the suction tube 112. In some embodiments, an outer surface of the projection portion 412 may abut the inner surface 220 of the distal segment 116 such that a fluid tight engagement is achieved between the hollow tip 114 and the suction tube 112. Thus, any leakage of fluid between the hollow tip 114 and the suction tube 112 is avoided. In some embodiments, the outer diameter of the projection portion 412 may be of 8 to 4 mm, preferably 7 to 4.5 mm, preferably 5 mm. In some embodiments, the outer diameter of the projection portion 412 may be constant throughout the length of the projection portion length ‘LP’. In alternative embodiments, cross-section of the projection portion 412 may be an oval, an ellipse or any other polygon shape known to one of ordinary skill in the art to engage with the distal end 120 of the suction tube 112. In some embodiments, the projection portion 412 has a cross-sectional shape that is the same as the cross-sectional shape of the suction tube 112. In alternative embodiments, the projection portion 412 has a cross-sectional shape that is not the same as the cross-sectional shape of the suction tube 112. As shown in FIG. 4, the projection portion length ‘LP’ is 10 mm and the outer diameter of the projection portion 412 is 5 mm.

In some embodiments, the elongated hollow body 402 of the hollow tip 114 further comprises a tapered portion 414 defined at the fixed end 406 and the free end 404. As such, the fixed end 406 of the hollow tip 114 comprises the projection portion 412 configured to reversibly connect with the distal end 120 of the suction tube 112 and the tapered portion 414 extending between the projection portion 412 and the free end 404. The tapered portion 414 facilitates entry of the hollow tip 114 into the ear canal 102 without causing any injuries or discomfort to the patient. The tapered portion 414 has an outer diameter that decreases moving along the longitudinal axis ‘LA3’ of the hollow tip 114 in a direction from the fixed end 406 towards the free end 404. In some embodiments, the tapered portion 414 may be replaced with a concave surface to cause smooth entry of the hollow tip 114 into the ear canal 102. With the free end 404 and the fixed end 406 having the projection portion 412 and the tapered portion 414, the hollow tip 114 is made devoid of sharp edges and/or points. Thus, the hollow tip 114 of the present disclosure is designed in such a way to insert into the ear canal 102 of the patient without causing any injuries or discomfort to the patient. In some embodiments, a tip of the free end 404 of the hollow tip 114 may be defined as a round edge or smooth blunt edge to avoid discomfort to the patient while inserting the ear suctioning device 110 in the ear canal 102. In some embodiments, the free end 404 may be made as soft part and the fixed end 406 may be made as semisolid part or hard part.

The hollow tip 114 further comprise a safety flange 416 which prevents the suction tube 112 from contacting the ear 100 or the ear canal 102 into which the ear suctioning device 110 is inserted. A schematic representation of the safety flange 416 is shown in FIG. 4 for mere purpose of illustration. In some embodiments, the safety flange 416 is disposed on the fixed end 406 of the hollow tip 114. The safety flange 416 may act as a stopper to prevent the suction tube 112 from contacting the ear 100 or the ear canal 102 when the hollow tip 114 is inserted into the ear canal 102. During the cleaning operation in the ear canal 102, the safety flange 416 may prevent contact of non-disposable part such as the suction tube 112 with the ear canal 102 thereby preventing contamination. Further, the safety flange 416 may help in effectively controlling transmission of infection from one ear to another ear of the same patient or from the ears of one patient to another patient as the non-disposable suction tube 112 is prevented from contacting the ear 100 or the ear canal 102 of the patient. In some embodiments, the safety flange 416 may be disposed in the fixed end 406 at a juncture between the projection portion 412 and the tapered portion 414. In such embodiments, an edge of the distal end 120 of the suction tube 112 may contact the safety flange 416 of the hollow tip 114 when the suction tube 112 and the hollow tip 114 are interlocked or engaged each other. In some embodiments, the safety flange 416 is disposed anywhere along a length of the tapered portion 414. In some embodiments, the safety flange 416 may be disposed on the tapered portion 414 adjacent to a juncture between the fixed end 406 and the free end 404. In such embodiments, the fixed end 406 of the hollow tip 114 is also prevented from contacting the ear 100 or the ear canal 112 of the patient. In some embodiments, the safety flange 416 may be adjustably or movably disposed on the fixed end 406 such that the operator may adjust a location of the safety flange 416 as per the convenience and comfort during the ear cleaning operation. In some embodiments, the safety flange 416 is a ring-shaped structure disposed on the fixed end 406 and extends radially with respect to the longitudinal axis ‘LA3’ of the hollow tip 114. In some embodiments, the safety flange 416 includes one or more elongated members extending radially with respect to the longitudinal axis ‘LA3’ of the hollow tip 114. In some embodiments, the safety flange 416 extends radially and perpendicularly with respect to the longitudinal axis ‘LA3’ of the hollow tip 114. In some embodiments, the safety flange 416 may extend radially and at an inclination with respect to the longitudinal axis ‘LA3’ of the hollow tip 114. In some embodiments, the safety flange 416 may be made of flexible materials such as silicone, elastomeric materials or any other flexible non-toxic materials known to one of ordinary skill in the art. In some embodiments, the safety flange 416 is made out of the same material as the free end 404, the fixed end 416, and/or the hollow tip 114. In some embodiments, the safety flange 416 may extend over, around, or encompass a portion of the distal end 120 of the suction tube 112.

In some embodiments, the hollow tip 114 comprises a visual size indicator 420. In some embodiments, the visual size indicator 420 acts as an identifier for the inner diameter of the free end 404. In some embodiments, a distinct visual size indicator is used for different sizes of the hollow tip. For example, one visual size indicator would correspond to the free end 404 having the outer diameter of 4 mm and the lumen 408 having the diameter of 3 mm, while another visual size indicator would correspond to the free end 404 having the outer diameter of 2.5 mm and the lumen 408 having the diameter of 1.5 mm. In the embodiment shown in FIG. 4, the visual size indicator is provided in the free end 404. The visual size indicator may be any suitable physical visual indicator known to one of ordinary skill in the art. Examples of such visual indicators include written marks, color marks, embossed or debossed marks, and engraved marks. The written marks, embossed or debossed marks and/or engraved marks may be alphanumeric characters or the equivalent thereof in any language of alphabet. The written marks may be disposed upon, attached to, or integrated into the hollow tip 114 using any suitable method known to one of ordinary skill in the art. Examples of such methods include, but are not limited to, printing onto the material of the hollow tip, printing onto a visual indicator material and affixing said material to the hollow tip, and printing into an embossed, debossed, or engraved mark. In some embodiments, the visual size indicator 420 comprises written or printed characters. In some embodiments, the visual size indicator 420 is a color mark. In embodiments where a collection of hollow tips of differing sizes as described above are provided, a unique visual size indicator is chosen for each differing size of the hollow tips. In some embodiments, the visual size indicator is provided in the free end 404 or the non-flexible fixed end 406. In some embodiments, the color mark may be yellow, red, blue, purple, orange, green or any other color that may help the operator to easily identify the size of the hollow tip 114. In some embodiments, the visual size indicator 420 is text corresponding to the outer diameter of the flexible free end 404 and/or the diameter of the lumen 408. Said text may be printed, embossed or debossed, or engraved on the free end 404 or the fixed end 406.

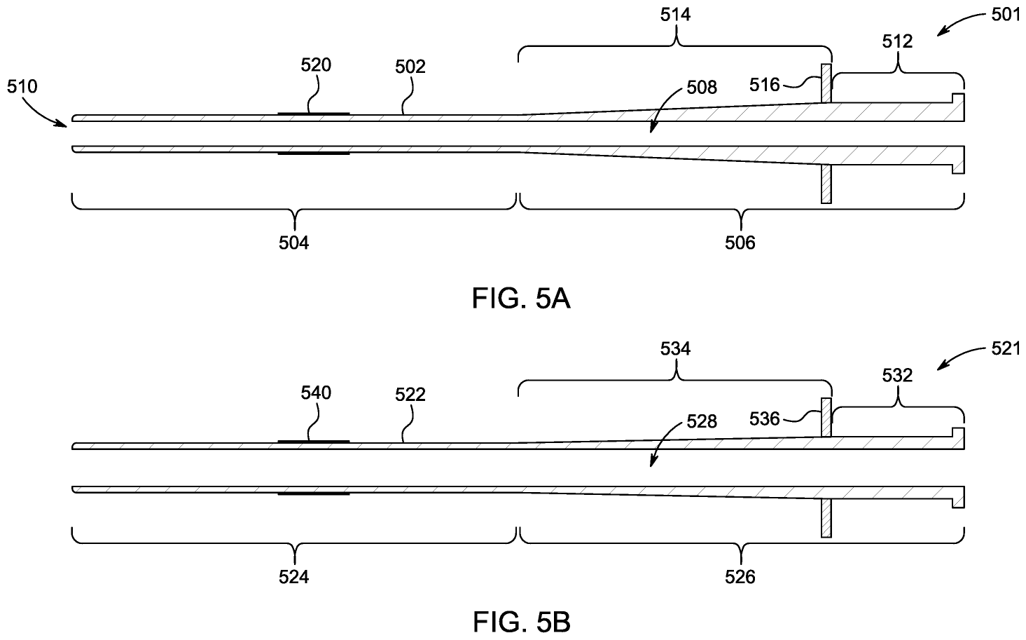

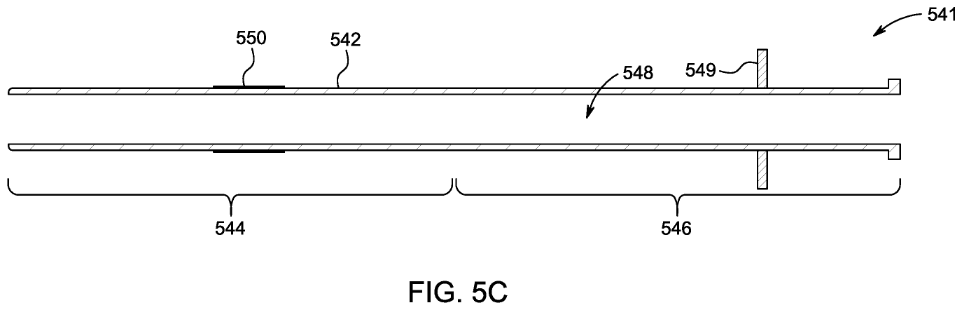

Referring to FIG. 5A, a longitudinal sectional view of a hollow tip 501 of the ear suctioning device 110 is illustrated, according to an embodiment of the present disclosure. The hollow tip 501 is reversibly and fluidly connected to the distal segment 116 of the suction tube 112. In some embodiments, the hollow tip 501 may include an elongated hollow body 502 having a free end 504 and a fixed end 506. The construction and dimensional specification, particularly a length of each of the free end 504 and the fixed end 506, and a total length of the hollow tip 501, are identical to the hollow tip 114. Further, the hollow tip 501 is made of materials identical to the materials used for making the hollow tip 114.

In some embodiments, the elongated hollow body 502 of the hollow tip 501 comprises a lumen 508 configured to align with the second lumen 214 of the distal segment 116 when the hollow tip 501 is engaged with the suction tube 112. The hollow tip 501 has a free end opening 510 having a free end opening inner diameter of 0.5 to 5 mm. Particularly, the free end opening 510 may be defined by the lumen 508. In some embodiments, the flexible free end 504 has an outer diameter which is constant throughout the length thereof. As shown in the exemplary embodiment depicted in FIG. 5A, the outer diameter of the free end 504 is 3 mm and the free end opening inner diameter or the diameter of the lumen 508 is 2 mm.

The construction and dimensional specification of the fixed end 506 having a projection portion 512 and a tapered portion 514 are identical to the construction and dimensional details of the fixed end 406 of the hollow tip 114. The hollow tip 501 comprise a safety flange 516 which prevents the suction tube 112 from contacting the ear 100 or the ear canal 102 into which the ear suctioning device 110 is inserted.

The hollow tip 501 comprises a visual size indicator 520. In some embodiments, the visual size indicator 520 may be a color mark. The color mark may correspond to the flexible free end 504 having the outer diameter of 3 mm and the lumen 508 having the diameter of 2 mm. In some embodiments, the color mark may be provided on the free end 504 or the fixed end 506. In some embodiments, the color mark may be red color or any other color that may help the operator to easily identify the size of the hollow tip 501. In some embodiments, the visual size indicator 520 may be a text corresponding to the outer diameter of the flexible free end 504 and the diameter of the lumen 508 embossed or engraved on the flexible free end 504 or the non-flexible fixed end 506. In some embodiments, the visual size indicator 520 may be the text printed on the free end 504 or the fixed end 506.

Referring to FIG. 5B, a longitudinal sectional view of a hollow tip 521 of the ear suctioning device 110 is illustrated, according to an embodiment of the present disclosure. The hollow tip 521 comprises an elongated hollow body 522 having a free end 524 and a fixed end 526. The construction and dimensional specification, particularly a length of each of the free end 524 and the fixed end 526, and a total length of the hollow tip 521, are identical to the hollow tips 114 and 501. Further, the hollow tip 521 is made of materials identical to the materials used for making the hollow tips 114 and 501. The elongated hollow body 522 of the hollow tip 521 comprises a lumen 528 configured to align with the second lumen 214 of the distal segment 116 when the hollow tip 521 is engaged with the suction tube 112. As shown in FIG. 5B, an outer diameter of the free end 524 is 4 mm and a diameter of the lumen 528 is 3 mm. The construction and dimensional specification of the fixed end 526 having a projection portion 532 and a tapered portion 534 are identical to the construction and dimensional details of the fixed ends 406 and 506 of the hollow tips 114 and 501, respectively. The hollow tip 521 comprise a safety flange 536 which prevents the suction tube 112 from contacting the ear 100 or the ear canal 102 into which the ear suctioning device 110 is inserted.

The hollow tip 521 comprises a visual size indicator 540. In some embodiments, the visual size indicator 540 may be a color mark. The color mark may correspond to the free end 524 having the outer diameter of 4 mm and the lumen 528 having the diameter of 3 mm. In some embodiments, the color mark may be provided on the free end 524 or the fixed end 526. In some embodiments, the color mark may be blue color or any other color that may help the operator to easily identify the size of the hollow tip 521. In some embodiments, the visual size indicator 540 may be a text corresponding to the outer diameter of the free end 524 and the diameter of the lumen 528 embossed or engraved on the free end 524 or the fixed end 526. In some embodiments, the visual size indicator 540 may be the text printed on the free end 524 or the fixed end 526.

Referring to FIG. 5C, a longitudinal sectional view of a hollow tip 541 of the ear suctioning device 110 is illustrated, according to an embodiment of the present disclosure. The hollow tip 541 comprises an elongated hollow body 542 having a free end 544 and a fixed end 546. The construction and dimensional specification, particularly a length of each of the free end 544 and the fixed end 546, and a total length of the hollow tip 541, are identical to the hollow tips 114, 501 and 521. Further, the hollow tip 541 is made of materials identical to the materials used for making the hollow tips 114, 501 and 521. The elongated hollow body 542 of the hollow tip 541 comprises a lumen 548 configured to align with the second lumen 214 of the distal segment 116 when the hollow tip 541 is engaged with the suction tube 112. As shown in FIG. 5C, an outer diameter of the free end 544 is 5 mm and a diameter of the lumen 548 is 4 mm. The hollow tip 541 comprise a safety flange 549 which prevents the suction tube 112 from contacting the ear 100 or the ear canal 102 into which the ear suctioning device 110 is inserted.

The hollow tip 541 comprises a visual size indicator 550. In some embodiments, the visual size indicator 550 may be a color mark. The color mark may correspond to the free end 544 having the outer diameter of 5 mm and the lumen 548 having the diameter of 4 mm. In some embodiments, the color mark may be provided on the free end 544 or the fixed end 546. In some embodiments, the color mark may be brown color or any other color that may help the operator to easily identify the size of the hollow tip 541. In some embodiments, the visual size indicator 550 may be a text corresponding to the outer diameter of the free end 544 and the diameter of the lumen 548 embossed or engraved on the free end 544 or the fixed end 546. In some embodiments, the visual size indicator 550 may be the text printed on the free end 544 or the fixed end 546. In some embodiments, the hollow tips 114, 501, 521, and 541 may be constructed as disposable components.

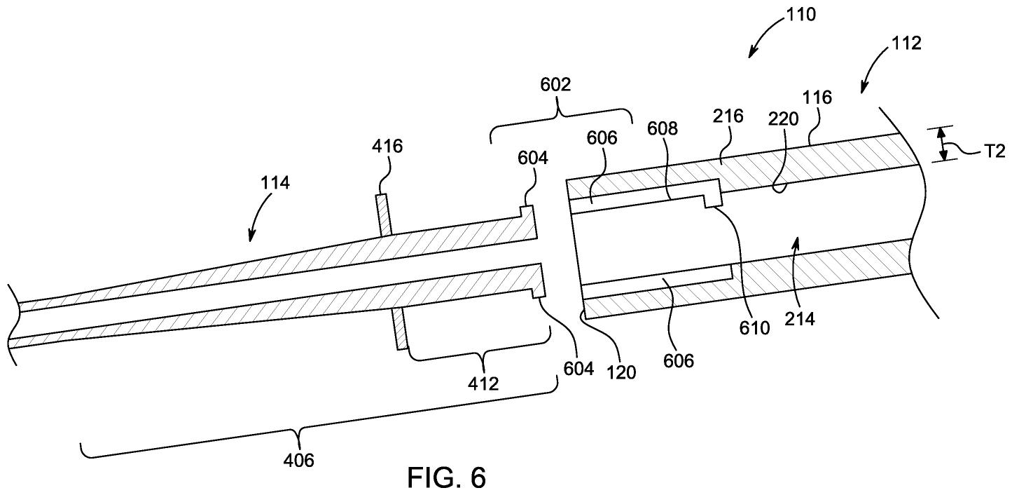

Referring to FIG. 6, a schematic diagram of a tip interlock 602 of the ear suctioning device 110 is illustrated, according to an embodiment of the present disclosure. The tip interlock 602 is explained with reference to the hollow tip 114 although the tip interlock 602 is similarly implemented on the hollow tips 501, 521 and 541. The tip interlock 602 comprises two or more interlock components and at least one of which is located on the suction tube 112 and at least one of which is located on the hollow tip 114. In some embodiments, the hollow tip 114 comprises one or more protrusions 604 extending radially away from the projection portion 412 of the fixed end 406. The protrusions 604 of the hollow tip 114 may be otherwise referred to as the interlock components of the hollow tip 114. In some embodiments, the protrusion 604 has a square cross-section. In some embodiments, cross-section of the protrusion 604 may be a circular, an oval, or any other polygon shape known to one of ordinary skill in the art. In some embodiments, the square cross-section of the protrusion 604 may have a side length of about 1 to 3 mm, preferably 1.25 to 2.75 mm, preferably 1.5 to 2.5 mm, preferably 1.75 to 2.25 mm. A thickness of the protrusion 604 extending from the projection portion 412 may be defined based on the thickness ‘T2’ of the wall 216 of the distal segment 116 of the suction tube 112. As shown in the exemplary embodiment depicted in FIG. 6, the hollow tip 114 includes two protrusions 604 which are located at the projection portion 412 diametrically opposite to each other. Further, the square cross-section of the protrusion 604 has a side length of 2 mm. In some embodiments, the two protrusions are not placed diametrically opposite to each other. In some embodiments, the hollow tip 114 includes more than two protrusions 604 located at the projection portion 412. In such embodiments, the protrusions may be equally or unequally spaced around a circumference or perimeter of the projection portion 412.

In some embodiments, the distal end 120 of the suction tube 112 comprises one or more grooves 606 defined at the inner surface 220 of the distal segment 116 of the suction tube 112. The one or more grooves 606 of the suction tube 112 may be otherwise referred to as the interlock components of the suction tube 112. A width of the groove 606 may be defined in conformance with the dimensional specification of the protrusion 604 of the hollow tip 112 such that the projection portion 412 of the hollow tip 114 may be slidably received within the distal segment 116 of the suction tube 112. Particularly, the width of the groove 606 may be defined based on the side length of the cross-section of the protrusion 604. Further, a depth of the groove 606 may be defined based on the thickness of the protrusion 604 of the hollow tip 114. As such, the number of grooves 606 and the dimensional specification of the grooves 606 may be defined in conformance with the number of protrusions 604 and dimensional specification of the protrusions 604. As shown in FIG. 6, the suction tube 112 comprises two grooves 606 corresponding to the two protrusions 604 provided on the hollow tip 114. In some embodiments, the groove 606 comprises a longitudinal portion 608 and a radial portion 610 extending perpendicularly from the longitudinal portion 608. In other words, the groove 606 is defined in the form of a L-shape. The radial portions 610 of the two grooves 606 extend from the longitudinal portions 608 in anti-clockwise direction hence the radial portion 610 of the bottom groove 606 is not visible in the sectional view as seen in FIG. 6. Alternatively, the radial portions 610 may extend in a clockwise direction. During interlocking process, the protrusions 604 of the hollow tip 114 are linearly received within the longitudinal portions 608 of the grooves 606 and further received within the radial portions 610 upon rotating or twisting the hollow tip 114 in the clockwise or anti-clockwise direction as appropriate. As such, the hollow tip 114 is interlocked with the distal segment 116 of the suction tube 112 and prevented from being accidentally detached from the suction tube 112. This mechanism may be referred to as a “twist-fit” mechanism. The hollow tips 501, 521 and 541 described with reference to FIG. 5A, FIG. 5B, and FIG. 5C, respectively, also include one or more protrusions identical to the one or more protrusions 604 of the hollow tip 114 to interlock with the distal end 120 of the suction tube 112.

In some embodiments, the tip interlock 602 comprises at least one interlock component located on the suction tube 112 and at least one interlock component located on the hollow tip 114 configured to achieve a snap-fit coupling mechanism. This snap-fit coupling mechanism is configured to facilitate reversible coupling between the hollow tip 114 and the suction tube 112. In some embodiments, the tip interlock 602 comprises at least one interlock component located on the suction tube 112 and at least one interlock component located on the hollow tip 114 to achieve a press-fit coupling mechanism therebetween. In some embodiments, the tip interlock 602 may comprise at least one interlock component located on the suction tube 112 and at least one interlock component located on the hollow tip 114 to achieve a quick release coupling mechanism therebetween.

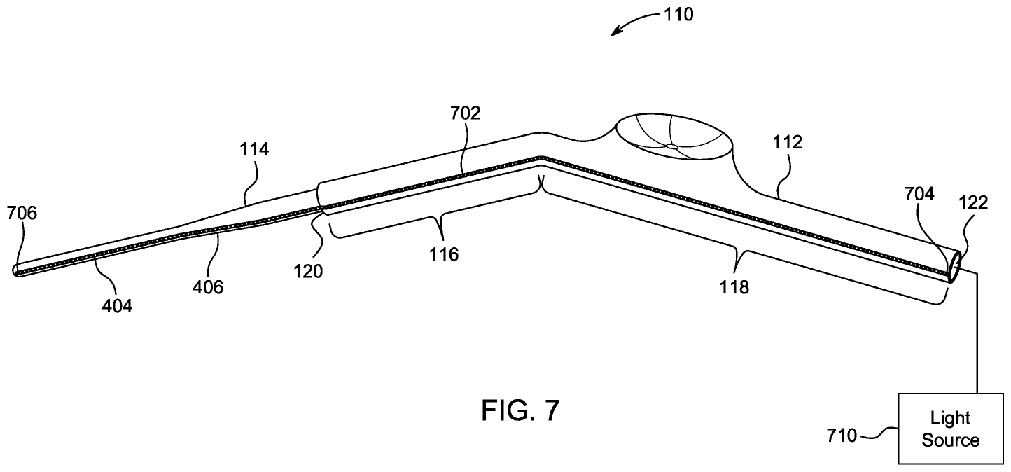

In some embodiments, the ear suctioning device 110 comprises a light guide. Referring to FIG. 7, a schematic perspective view of the ear suctioning device 110 showing an exemplary light guide 702 is illustrated, according to an embodiment of the present disclosure. The light guide 702 is depicted substantially oriented along a longitudinal axis of the suction tube 112, particularly along the first longitudinal axis ‘LA1’ of the proximal segment 118 and the second longitudinal axis ‘LA2’ of the distal segment 116, and the longitudinal axis ‘LA3’ of the hollow tip 114. In general, the light guide acts to direct illumination from an illumination source to the ear or ear canal of a patient undergoing suction with the ear suctioning device 110. The light guide may be any suitable structure or material for achieving this purpose. Preferably, the light guide is made of an optically transparent material. In some embodiments, the light guide 702 is a thin filament. This filament may be made of transparent material that may transmit light signal through successive internal reflections. In some embodiments, the light guide 702 is constructed using flexible or non-flexible material. In some embodiments, the light guide 702 may be an optical fiber. This optical fiber may be made of plastic, glass, or any other suitable material known to one of ordinary skill in the art.

In some embodiments, the light guide is attached to, disposed upon, or integrated with the proximal segment 118, the distal segment 116 and/or the hollow tip 114. In some embodiments, the light guide 702 may be separately attached to the suction tube 112 and the hollow tip 114. In such embodiments, the light guide 702 may be attached to the ear suctioning device 110 upon interlocking the hollow tip 114 and the suction tube 112. In some embodiments, the light guide 702 may be attached to an outer surface of the suction tube 112 and the hollow tip 114. In such embodiments, the light guide 702 may be attached to the suction tube 112 and the hollow tip 114 using an adhesive, fastening mechanisms such as snap-fit, or any other attaching mechanisms known to one of ordinary skill in the art to detachably attach the light guide 702 on the suction tube 112 and the hollow tip 114. In some embodiments, the light guide 702 may be integral to the suction tube 112 and the hollow tip 114. In such embodiments, the hollow tip 114 is aligned with the suction tube 112 with respect to the light guide 702 while interlocking the hollow tip 114 and the suction tube 112. Further, the light guide 702 associated with each of the hollow tip 114 and the suction tube 112 may be connected to each other. In some embodiments, the light guide 702 may be disposed within the interior volume 130 defined by the first lumen 204 and the second lumen 214 of the suction tube 112 and the lumen 408 of the hollow tip 114.

In such embodiments, the light guide 702 may be embedded at an inner surface of the lumens of the suction tube 112 and the hollow tip 114.

The light guide 702 comprises a source end 704 and an illumination end 706. The source end 704 may be disposed adjacent to the proximal end 122 of the suction tube 112 and the illumination end 706 may be disposed adjacent to the free end 404 of the hollow tip 114. In some embodiments, the light guide 702 may be disposed in the suction tube 112 and substantially oriented along the first longitudinal axis ‘LA1’ of the proximal segment 118 and the second longitudinal axis ‘LA2’ of the distal segment 116. In such embodiments, the source end 704 of the light guide 702 may be disposed adjacent to the proximal end 122 and the illumination end 706 may be disposed adjacent to the distal end 120 of the suction tube 112. Further, the light guide 702 may be made of flexible or non-flexible material. In some embodiments, the light guide 702 may be disposed in the hollow tip 114 and substantially oriented along the longitudinal axis ‘LA3’ thereof. In such embodiments, the source end 704 of the light guide 702 may be disposed on the non-flexible fixed end 406 of the hollow tip 114 and the illumination end 706 may be disposed near the tip of the free end 404 of the hollow tip 114. Further, the light guide 702 may be made of flexible material to provide flexibility in the construction of the hollow tip 114. In some embodiments, the illumination end 706 may be disposed adjacent a location of the safety flange 416.

In some embodiments, the ear suctioning device 110 further comprises a light source 710. This light source 710 is configured to provide illumination to the source end 704 of the light guide 702. In some embodiments, the light source 710 may be an electrical light source connected to the source end 704 of the light guide 702, for example an LED. In some embodiments, the light source 710 may be a battery powered electrical light source configured to provide illumination to the source end 704 of the light guide 702. In some embodiments, the light source 710 may be reversibly attached to the source end 704 of the light guide 702.

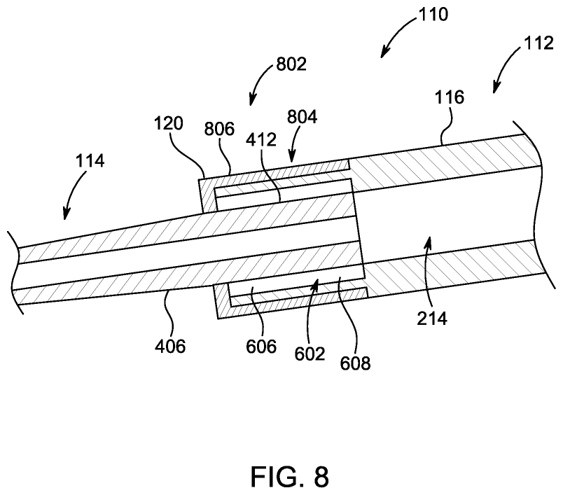

In some embodiments, the ear suctioning device 110 further comprises a hollow tip ejection mechanism 802. Referring to FIG. 8, a schematic diagram of a hollow tip ejection mechanism 802 of the ear suctioning device 110 is illustrated, according to an embodiment of the present disclosure. The hollow tip ejection mechanism 802 of the ear suctioning device 110 is explained with reference to FIG. 6 and FIG. 8. Referring to FIG. 6 and FIG. 8, the tip interlock 602 of the ear suctioning device 110 comprises the at least one interlock component, such as the one or more protrusions 604 extending radially away from the projection portion 412 of the fixed end 406, located on the hollow tip 114 and the at least one interlock component, such as the one or more grooves 606, located on the suction tube 112. As such, the hollow tip 114 is interlocked with the distal end 120 of the suction tube 112 using the tip interlock 602 and prevented from being accidentally detached from the suction tube 112. In some embodiments, the hollow tip ejection mechanism 802 may include one or more additional interlock components, which may be otherwise referred to as ‘the releasing mechanism 804’, at the distal end 120 of the suction tube 112. As such, with the releasing mechanism 804, the hollow tip ejection mechanism 802 may allow the hollow tip 114 to be disconnected and removed from the suction tube 112 without the user, or the operator, making contact with the hollow tip 114. In such embodiments, the releasing mechanism 804 may be actuated by the operator to disconnect or disengage the one or more protrusions 604 from the one or more corresponding grooves 606 and remove the hollow tip 114 from the suction tube 112.

A schematic representation of the exemplary releasing mechanisms 804 is shown in FIG. 8 for the mere purpose of illustration. In some embodiments, the releasing mechanism 804 may include a cylindrical body 806 rotatably engaged with the distal end 120 of the suction tube 112 and detachably coupled with the hollow tip 114 when the hollow tip 114 is interlocked with the suction tube 112. While removing the hollow tip 114 from the suction tube 112, the cylindrical body 806 of the releasing mechanism 804 may be rotated relative to the distal end 120 of the suction tube 112. As the cylindrical body 806 is engaged with the hollow tip 114, the hollow tip 114 also rotates along with the cylindrical body 806 and the one or more protrusions 604 of the hollow tip 114 may come out of the radial portions 610 of the one or more grooves 604 and align with the longitudinal portions 608 of the grooves 606. Further, the ear suctioning device 110 may be held to position the hollow tip 114 downward such that the one or more protrusions 604 of the hollow tip 114 may slide through the longitudinal portions 608 of the grooves 606 due to gravity and the hollow tip 114 may be then removed from the suction tube 112 without the operator touching the hollow tip 114.

In some embodiments, the releasing mechanism 804 may include a spring member (not shown), for example a torsional spring, attached to the cylindrical body 806 and the distal end 120 of the suction tube 112. As such, the cylindrical body 806 may be biased in a non-actuated position with the help of the spring member and moved to an actuated position against a biasing force of the spring member. Thus, the cylindrical body 806 is normally held in the non-actuated position and return to the non-actuated position due to the biasing force even after the cylindrical body 806 is moved to the actuated position. In the non-actuated position of the cylindrical body 806, the hollow tip ejection mechanism 802 may facilitate reversible connection of the hollow tip 114 with the suction tube 112, and in the actuated position, the cylindrical body 806 allows removal of the hollow tip 114 from the suction tube 112.

In some embodiments, the hollow tip ejection mechanism 802 may include a sliding button coupled to, disposed on, or integrated with the distal end 120 of the suction tube 112 and engaged with the fixed end 406 of the hollow tip 114. The sliding button may move relative of the distal end 120 of the suction tube 112 and allow the hollow tip 114 to be disconnected and removed from the suction tube 112 without the operator touching the hollow tip 114. In some embodiments, the sliding button may move along the second longitudinal axis ‘LA2’ of the distal segment 116 of the suction tube 112. In some embodiments, the sliding button may rotate clockwise and anti-clockwise relative to the distal end 120 of the suction tube 112. In some embodiments, the hollow tip ejection mechanism 802 may include a press button located at the distal end 120 of the suction tube 112 and may be configured to disconnect and remove the hollow tip 114 from the suction tube 112 without the operator making contact with the hollow tip 114. The sliding button may be used to decouple press-fit or snap-fit mechanisms which interconnect the hollow tip 114 and the suction tube 112.

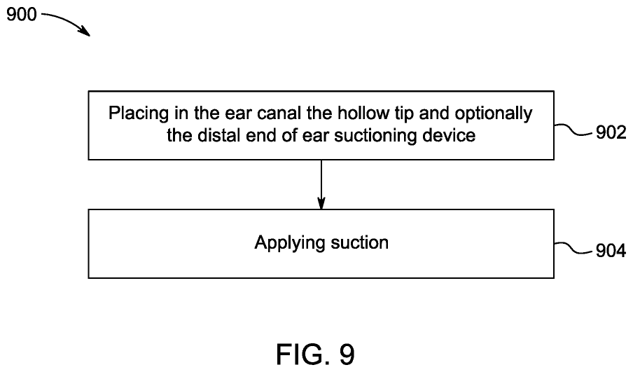

Referring to FIG. 9, a flow diagram of a method 900 of providing suction to the ear canal 102 of the patient is illustrated, according to an embodiment of the present disclosure. The method 900 of providing suction to the ear canal 102 is illustrated with reference to FIG. 1 through FIG. 8, and particularly with the hollow tip 114 described in FIG. 4. At step 902, the method 900 includes placing the hollow tip 114, and optionally the distal end 120 of the ear suctioning device 110, in the ear canal 102 of the patient. For cleaning the ear canal 102 of the patient such as removing wax, debris, fluids, discharge, foreign objects, and other contaminants, the hollow tip 114 of the present disclosure is reversibly connected to the distal segment 116 of the suction tube 112. Particularly, the fixed end 406 of the hollow tip 114 is reversibly engaged with the distal end 120 of the suction tube 112. The ergonomic grip 224 provided on the proximal segment 118 of the suction tube 112 facilitates the operator to firmly hold the suction tube 112. The operator may select an appropriate hollow tip from the various hollow tips 114, 501, 521, and 541 described in FIGS. 4, 5A, 5B, and 5C, respectively, based on factors such as the diagnostic condition of the ear canal 102 of the patient and the age of the patient. The visual size indicators 420, 520, 540, and 550 provided on the hollow tips 114, 501, 521, and 541, respectively, will facilitate the operator to choose suitable hollow tip for the ear cleaning operation. These visual size indicators may be advantageous for reducing user error and increasing patient safety by allowing quick and facile identification of the tip size. Upon the ear suctioning device 110 is prepared for the cleaning operation, the proximal end 122 of the suction tube 112 is attached to the suction generation device 124 through the vacuum line 126. Then the hollow tip 114 of the ear suctioning device 110 is placed in the ear canal 102 of the patient. In some circumstances, the distal end 120 of the suction tube 112 may be optionally placed just outside the ear canal 102. In some embodiments, the safety flange 416 provided on the hollow tip 114 may prevent the suction tube 112 from contacting the ear 100 or the ear canal 102 of the patient.

At step 904, the method 900 includes applying suction in the ear suctioning device 110. The suction generation device 124 connected to the proximal end 122 of the suction tube 112 is used for applying suction to the ear suctioning device 110. The phrase ‘applying suction’ may be defined as creating vacuum or region of lower-than-ambient air pressure within the interior volume 130 of the suction tube 112. In other words, air pressure within the interior volume 130 of the suction tube 112 may be reduced. At the step 904, the suction generation device 124 may be operated to reduce air pressure within the interior volume 130 of the suction tube 112. The method 900 further comprises reversibly occluding the pressure-equalizing hole 128 to adjust a suction strength applied to the ear canal 102. Particularly, the operator may totally or partially occlude the pressure-equalizing hole 128 to further control the air pressure within the interior volume 130 of the suction tube 112. The suction strength may be defined as the vacuum pressure created or achieved within the interior volume 130 of the suction tube 112 sufficient enough to extract the wax, debris or other foreign objects from the ear canal 102. A higher suction strength may be achieved when the pressure-equalizing hole 128 is totally occluded and the suction strength diminishes when the pressure-equalizing hole 128 is partially occluded. In some embodiments, the operator may use the finger or the thumb 310 to totally or partially occlude the pressure-equalizing hole 128 defined in the finger cup 302 to adjust the air pressure within the interior volume 130. In some embodiments, the reversible occluding may be defined as total or partial occluding of the pressure-equalizing hole 128 using the finger or the thumb 310 of the operator. Further, the vacuum pressure within the interior volume 130 of the suction tube 112 may be adjusted or controlled using the pressure-equalizing hole 128 while keeping the ear suctioning device 110 in the ear 100 or the ear canal 102 and without adjusting any operating settings of the suction generation device 124. In some embodiments, the operator may use the controllable occlusion mechanism 312 to adjust the air pressure within the interior volume 130. Thus, the air pressure within the interior volume 130 of the suction tube 112 is controlled to remove the wax, debris or other foreign objects from the ear canal 102. The vacuum created within the interior volume 130 may extract the wax, debris and other foreign objects therethrough. The performance and efficiency of the ear cleaning operation may be defined based on various factors including, but not limited to, the suction strength created within the interior volume 130 and the type of hollow tip used. The method involving placing the hollow tip 114, and optionally the distal end 120 of the ear suctioning device 110, in the ear canal 102 of the patient and applying suction may be referred to as an “ear cleaning” or “ear cleaning procedure”. In preferred embodiments, the method further comprises removing the hollow tip 114 from the suction tube 110. In such embodiments, the removing is preferably performed at a conclusion of or after the ear cleaning procedure. A hollow tip 114 which has been used in an ear cleaning procedure may be referred to as a “used tip”, particularly after removing. The ear suction device, following the removing, may be referred to as a “tipless ear suctioning device”. In some embodiments, the removing involves deinterlocking the hollow tip 114 from the suction tube 110. In some embodiments, the removing involves ejection of the hollow tip 114 using the hollow tip ejection mechanism 802. In preferred embodiments, following being removed, the hollow tip 114 is disposed of. In such embodiments, the hollow tip 114 is preferably disposed of into a suitable medical waste container. In some embodiments, the hollow tip 114 is disposed of following any suitable protocol or method for handing and/or disposing of hazardous or non-hazardous medical waste known to one of ordinary skill in the art. In some embodiments, a single hollow tip 114 is used to clean both ears of a single patient. In alternative embodiments, a single hollow tip 114 is used to clean only one ear of a single patient. In such embodiments, a fresh hollow tip 114 is used for each ear of a single patient. A “fresh” hollow tip, as used herein, refers to a hollow tip 114 which has not been used for ear cleaning, has not been in contact with the skin of any human, is sterile, or a combination of these.

The ear suctioning device 110 of the present disclosure helps in effectively controlling transmission of infection from one ear to another ear of the same patient or from the ears of one patient to another patient. Further, occupational hazards and infections are controlled inside the otorhinolaryngology clinics. Decreases the incidence of chronic ear diseases, hearing loss, vertigo and tinnitus which are otherwise caused by iatrogenic traumas. Helps in minimizing the need of ear syringing and thus avoid the complications of ear syringing and irrigation. Further, ear canal wall injuries are minimized or prevented which otherwise caused by iatrogenic suction tube trauma. Overcome the frequent blockages which otherwise caused by the existing suction tubes. As frequent plugging is not required, maintenance of the ear suctioning device 110 is decreased. Due to the simple construction and ease of handling, junior or senior physician can operate the device without any training requirement. The interlock tip 602 and the visual size indicators 420, 520, 540, and 550 help the operator to easily and quickly identify the hollow tips 114, 501, 521, and 541, respectively, and interlock with the suction tube 112. The hollow tip ejection mechanism 802 helps the operator to easily and quickly replace or remove the hollow tip 114 from the suction tube 112. The ear suctioning device 110 further helps in training junior physicians on microsuction.

The ear suctioning device is very practical in poor communities and area of crises when there are no sufficient sterilization facilities. Further, the ear suctioning device 110 may be used in nasal suctioning and neurosurgical procedures. Further, microsuction procedure in operating room, hospital admission and complications of general anesthesia may be avoided. Thus, the ear suctioning device 110 of the present disclosure renders a cost effective and simple solution in the otorhinolaryngology clinics for the cleaning of the ear 100 and the ear canal 102.

The examples below are intended to further illustrate protocols for and are not intended to limit the scope of the claims.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

1-2. (canceled)

3. The method of claim 18, wherein the ear suctioning device further comprising an ergonomic grip located on or attached to the proximal segment.

4-5. (canceled)

6. The method of claim 18, wherein the ear suctioning device further comprising a tip interlock comprising two or more interlock components, at least one of which is located on the suction tube and at least one of which is located on the hollow tip.

7. The method of claim 18, wherein the hollow tip of the ear suctioning device comprises a flexible free end and a non-flexible fixed end.

8-17. (canceled)

18. A method of providing suction to an ear canal, comprising:
placing in the ear canal a hollow tip of an ear suctioning device;
wherein the ear suctioning device comprises:
a suction tube having a distal segment and a proximal segment which are angled with respect to each other at about 130 to 170 degrees;
the hollow tip reversibly connected to a distal end of the suction tube, the hollow tip being fluidly connected to the suction tube; and
a pressure-equalizing hole disposed on the proximal segment, the pressure-equalizing hole being in fluid communication with an interior volume of the suction tube, wherein the pressure-equalizing hole of the ear suctioning device is located in a finger cup having a substantially hemiovoid shape and is configured such that the pressure-equalizing hole is capable of being partially or totally occluded by a finger or thumb placed in the finger cup and the finger cup is elevated from an outer surface of the suction tube and the pressure-equalizing hole extends from a center point of the hemiovoid shape to the interior volume of the suction tube;
wherein a proximal end of the suction tube is configured to attach to a vacuum or low pressure line; and
the hollow tip comprises a safety flange which prevents the suction tube from contacting an ear or ear canal into which the ear suctioning device is inserted; and
establishing a reduced pressure inside the ear suction device by applying suction through a proximal end of the suction tube of the ear suctioning device;
at least partially occluding the pressure-equalizing hole by manually positioning a finger surface over or around the pressure-equalizing hole to cause ear fluid to flow from the ear canal through the hollow tube into the suction tube.

19. The method of claim 18, wherein the method further comprises reversibly occluding the pressure-equalizing hole to adjust a suction strength applied to the ear canal, the occluding being partial occluding or total occluding.

20. The method of claim 19, wherein the method further comprises:
removing from the ear suctioning device the hollow tip to form a tipless ear suctioning device and a used tip; and
disposing of the used tip.

21. (canceled)

22. The method of claim 18, wherein the ear suctioning device further comprises:
a controller and a suction generation device,
wherein the controller is in communication with a sensor that is indicative of a vacuum pressure within an interior volume of the suction tube and the suction generation device is connected to the suction tube,
wherein the controller determines a value of the vacuum pressure based on a signal received from the sensor to actuate a controllable occlusion mechanism to partially or totally occlude the pressure-equalizing hole based on the value of the vacuum pressure.
